(19) United States
(12) Patent Application Publication
(10) Pub. No.: US 2022/0349894 A1
(43) Pub. Date: Nov. 3, 2022

(54) Peptide Fragment, Monoclonal Antibody, Colloidal Gold Test Strip And Detection Method Thereof Used For Detection Of Stichopus Oligopeptide

(71) Applicant: DALIAN DEEP BLUE PEPTIDE TECHNOLOGY RESEARCH AND DEVELOPMENT CO., LTD., Dalian (CN)

(72) Inventors: Weiyang BAO, Dalian (CN); Zuzhe WANG, Dalian (CN); Aihua ZUO, Dalian (CN); Pu MA, Dalian (CN); Tianli SUN, Dalian (CN); Linying ZHAO,, Dalian (CN)

(21) Appl. No.: 17/418,268

(22) PCT Filed: Sep. 8, 2020
(86) PCT No.: PCT/CN2020/113918

§ 371 Date: Jun. 25, 2021

(30) Foreign Application Priority Data
Jan. 14, 2020 (CN) 202010038879.4

(51) Int. Cl. G01N 33/68; C07K 14/435; G01N 33/543

(52) U.S. Cl. CPC G01N 33/68; C07K 14/43504; G01N 33/54388

(57) ABSTRACT

The invention belongs to the field of biotechnology, in particular, relates to peptide fragment, monoclonal antibody, colloidal gold test strip and detection method thereof used for detection of stichopus oligopeptide. Sequences of the peptide fragments are KIVPGVPD and GRDGDQGPV. After identification, the peptides KIVPGVPD and GRDGDQGPV are newly discovered oligopeptides, which are exclusive peptide fragments of Stichopus oligopeptide.

### REFERENCE TO SEQUENCE LISTING SUBMITTED VIA EFS-WEB

The entire content of the following electronic submission of the sequence listing via the USPTO EFS-WEB server, as authorized and set forth in MPEP § 1730 II.B.2(a)(C), is incorporated herein by reference in its entirety for all purposes. The sequence listing is identified on the electronically filed text file as follows:

### TECHNICAL FIELD

The invention belongs to the field of biotechnology, in particular, relates to peptide fragment, monoclonal antibody, colloidal gold test strip and detection method thereof used for detection of stichopus oligopeptide.

### BACKGROUND

Holothurian belongs to Echinodermata, holothurian class and Scutellara animals. It is a rare nutritional and health food with high protein, low fat, low sugar and extremely low cholesterol in the world, and has extremely high nutritional value and medicinal value. Compared with different varieties of holothurians, the contents of protein, polysaccharide and minerals are quite different. There are about 1200 species of holothurians in the world, but most of them have no edible value. According to statistics, there are about 40 kinds of edible holothurians in the world, while there are only about 20 kinds of edible holothurians in China, and only a few kinds of holothurians have high commercial value.

Stichopus is a very important economic aquatic animal in China, which is not only rich in nutrition, but also has reasonable nutrient composition. It is a kind of seafood which is deeply loved by people and has high nutritional value. The stichopus oligopeptide is obtained from sea cucumbers as raw materials by enzymatic hydrolysis with biological enzymes, which has the advantages of small molecular weight, easy absorption and high bioavailability.

In recent years, a variety of oligopeptide products have appeared on the market, all of which are white or yellowish powder. It is impossible to identify the source of oligopeptides with naked eyes or rapid and effective methods. Due to the lack of effective methods to identify stichopus oligopeptide at present, many unscrupulous merchants use sea eggplant, Panax ginseng and other raw materials to prepare oligopeptides that are shoddy, and even use peptides prepared by other high-protein animals and plants as stichopus oligopeptide to deceive consumers. Therefore, an efficient and accurate method for identifying stichopus oligopeptide is needed.

### SUMMARY

According to the shortcomings and requirements in the above fields, the invention provides peptide fragment, monoclonal antibody, colloidal gold test strip and detection method thereof used for detecting stichopus oligopeptide.

The technical scheme of the invention for protection is as follows:

One of the objects of the present invention is to provide a peptide fragment for preparing a colloidal gold test strip for detecting stichopus oligopeptide, and the amino acid sequences of the peptide fragment are KIVPGVPD and GRDGDQGPV.

A second object of the present invention is to provide a specific anti-KIVPGVPD monoclonal antibody obtained by immunizing animals with a conjugate KIVPGVPD-BSA obtained by coupling the KIVPGVPD peptide fragment of claim 1 with bovine serum albumin as an antigen.

Further, the preparation method of the monoclonal antibody comprises the following steps:

Solution 1 is obtained by dissolving PBS in 0.01 mol/L PBS at pH 7.4; solution 2 is obtained by dissolving KIVPGVPD peptide fragment in 0.01 mol/L PBS at pH7.4; solution 3 is obtained by dissolving 4 mg of 1-(3-dimethylaminopropyl)-3-ethylcarbodiimide hydrochloride in 0.01 mol/L PBS at pH 7.4;

Add solution 2 into solution 1, mix evenly, ice bath for 30 min, centrifuge to remove precipitation, add supernatant droplets into solution 3, continue ice bath for 15 min, and stir at room temperature for 5 min; Dialysis in 0.01 mol/L PBS at pH7.4 for 24 h; During this period, change the dialysate twice, and carry out centrifugation to remove precipitation to obtain the conjugate KIVPGVPD-BSA of the peptide fragment KIVPGVPD and bovine serum albumin; Immunize BALB/C mice with KIVPGVPD-BSA; The spleen lymphocytes of immunized mice and myeloma cell SP2/0 is fused under the promotion of PEG, and the hybridoma cell line stably secreting KIVPGVPD-BSA monoclonal antibody is screened; The hybridoma cell line is grown in increments, and the obtained culture solution is purified by the caprylic acid-saturated ammonium sulfate method to obtain monoclonal antibodies.

The third object of the present invention is to provide a specific anti-GRDGDQGPV monoclonal antibody obtained by immunizing animals with the conjugate GRDGDQGPV-BSA obtained by coupling the GRDGDQGPV peptide fragment with bovine serum albumin as an antigen.

The fourth purpose of the invention is to provide a colloidal gold test strip prepared by using the monoclonal antibody for detecting stichopus oligopeptide, wherein it comprises a sample pad, a gold label pad, an NC film, an absorbent pad and a PVC bottom plate;

The sample pad, the gold label pad, the NC film and the absorbent pad are sequentially arranged on the PVC bottom plate according to the sample chromatography direction; The gold label pad is coated with the monoclonal antibody labeled with colloidal gold;

The NC membrane sequentially comprises a detection line and a quality control line according to the direction of sample chromatography, wherein the detection line is coated with a conjugate of a peptide fragment and bovine serum albumin, and the quality control line is coated with goat anti-mouse secondary antibody.

Further, the monoclonal antibody labeled with colloidal gold is prepared according to the following method:

Take 100 ml of 0.01% chloroauric acid solution and heat it, add 2 ml of 1% trisodium citrate after boiling, and continue to boil for 5 minutes after the solution turns to wine red. After cooling to room temperature, make up to 100 ml with ultrapure water to obtain a colloidal gold solution with a gold particle diameter of about 10 nm. Adjust the pH value of the obtained 100 ml colloidal gold solution to 8.2 with 20 mol/L borate buffer, stir the colloidal gold solution and slowly add 45 μg of monoclonal antibody per ml of colloidal gold solution, react for 30 minutes, add 10% BSA in volume fraction to a final concentration of 1% in volume fraction, and stir for 10 minutes, centrifuge at 45,000 rpm for 30 min at 4° C., discard the supernatant and the resulting precipitate is the purified colloidal gold-labeled monoclonal antibody.

The fifth purpose of the present invention is to provide a method for detecting stichopus oligopeptide by using the colloidal gold test strip, wherein it comprises the following steps:

S1. Dissolve the sample with distilled water and add 3-4 drops onto the sample pad;

S2. After the quality control line develops color, observe whether the detection line changes from colorless to red; If the detection line turns red, the sample to be tested is not stichopus oligopeptide; If the detection line does not change color, the sample to be tested is stichopus oligopeptide.

The invention provides a peptide fragment, monoclonal antibody, colloidal gold test strip and detection method thereof used for detecting stichopus oligopeptide. The principle is: After the sample is dropped onto the sample pad, it swims in the direction of the absorbent pad, the colloidal gold-labeled monoclonal antibody (gold-labeled antibody) against KIVPGVPD (or GRDGDQGPV) peptide on the gold label pad is dissolved. If the sample is not stichopus oligopeptide, then when the gold-labeled antibody reaches the detection line on the NC membrane, the gold-labeled antibody is captured by the KIVPGVPD-BSA (or GRDGDQGPV-BSA) conjugate coated on the detection line, and deposited to turn the detection line red. The excess gold-labeled antibody continues to swim forward and is captured by the goat anti-mouse secondary antibody on the quality control line, making the quality control line red. If the sample is stichopus oligopeptide, the gold-labeled antibody combines with the peptide fragment KIVPGVPD (or GRDGDQGPV) and swims forward, crosses the detection line, and is captured by goat anti-mouse secondary antibody on the quality control line, making the quality control line turn red while the detection line does not change color. Only when the quality control line is red, the detection result is effective. The method of the invention has high accuracy, with a detection sensitivity of 20 g/ml, good specificity, easy observation of results, and can realize rapid screening of large quantities of samples.

### BRIEF DESCRIPTION OF DRAWINGS

FIG. 1 is a structural schematic diagram of a colloidal gold test strip prepared by using specific peptide fragments of the present invention.

In which: 1. Sample pad; 2. gold label pad; 3. NC membrane; 4. detection line; 5. quality control line; 6. absorbent pad; 7. PVC bottom plate.

FIG. 2 is a schematic diagram of the detection results of the colloidal gold test strip prepared by using specific peptide fragments of the present invention.

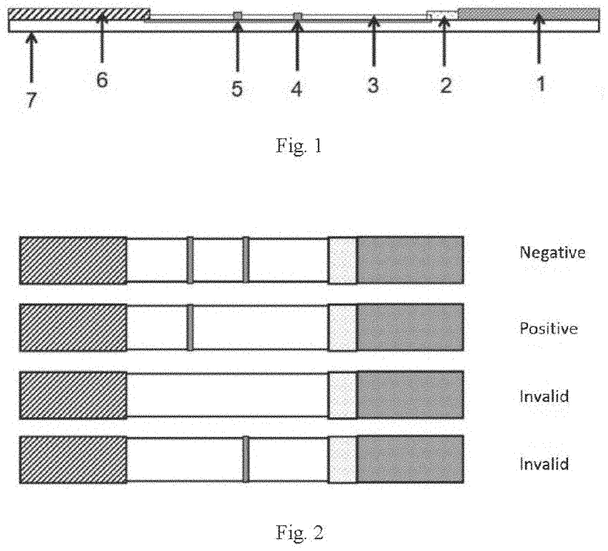

### DESCRIPTION OF THE EMBODIMENTS

The invention is described in detail in combination with the drawings and specific embodiments, but it should not be understood as a limitation of the invention. Unless otherwise specified, the technical means used in the following embodiments are conventional means well known to those skilled in the art, and the materials and reagents used in the following examples can be obtained from commercial sources unless otherwise specified.

### Embodiment 1

Sequence Source and Alignment Information of Specific Peptide Fragments

1. Sources of Stichopus-Specific Oligopeptide KIVPGVPD

Stichopus-specific oligopeptide KIVPGVPD, whose amino acid sequence is as shown in SEQ ID NO. 1, is derived from protein 2 alpha fibrillar collagen, whose Accesion number on NCBI is PIK60694, and whose amino acid sequence is as shown in SEQ ID NO. 2. After NCBI blast comparison, the peptide fragment was not consistent with any other species, and it is further identified as a new oligopeptide by online database BIOPEP and EROP-Moscow.

2. Sources of Stichopus-specific oligopeptide GRDGDQGPV

Stichopus-specific oligopeptide GRDGDQGPV has an amino acid sequence as shown in SEQ ID NO. 3 and is derived from protein alpha-2 collagen. Its Accesion number on NCBI is PIK60696, and its amino acid sequence is shown in SEQ ID NO. 4. After NCBI blast comparison, the peptide fragment was not consistent with any other species, and it is further identified as a new oligopeptide by online database BIOPEP and EROP-Moscow.

### Embodiment 2

Preparation of Colloidal Gold Test Strip

1. Colloidal Gold Test Strip Prepared by Peptide Fragment KIVPGVPD

Dissolve 10 mg of BSA in 0.01 mol/L PBS (pH 7.4)(solution 1). Dissolve 4 mg of purified KIVPGVPD peptide fragment in 0.01 mol/L PBS (pH 7.4)(solution 2). Dissolve 4 mg of 1-(3-dimethylaminopropyl)-3-ethylcarbodiimide hydrochloride (EDC) in 0.5 ml of 0.01 mol/L PBS (pH 7.4)(solution 3). Add solution 2 to solution 1, mix well in ice bath for 30 min, centrifuge to remove precipitation, add supernatant droplets into solution 3, continue ice bath for 15 min, and stir at room temperature for 5 min. Dialysis in 0.01 mol/L PBS (pH7.4) for 24 h, during this period, change the dialysate twice, and carry out centrifugation to remove precipitation to obtain the conjugate of the peptide fragment KIVPGVPD and bovine serum albumin; Immunize BALB/C mice with KIVPGVPD-BSA; The spleen lymphocytes of immunized mice and myeloma cell (SP2/0) is fused under the promotion of PEG, and the hybridoma cell line stably secreting KIVPGVPD-BSA monoclonal antibody is screened; The hybridoma cell line is grown in increments, and the obtained culture solution is purified by the caprylic acid-saturated ammonium sulfate method to obtain monoclonal antibodies, which is frozen and stored at −20° C. for later use.

Put an Erlenmeyer flask containing 100 ml of 0.01% chloroauric acid solution on a magnetic stirrer to heat and stir. Quickly add 2 ml of 1% trisodium citrate after boiling, and continue to boil for 5 minutes after the solution turns to wine red. After cooling to room temperature, make up to 100 ml with ultrapure water to obtain a colloidal gold solution with a gold particle diameter of about 10 nm. Adjust the pH value of the obtained 100 ml colloidal gold solution to 8.2 with 20 mol/L borate buffer, slowly add 45 μg of monoclonal antibody KIVPGVPD-BSA per ml of colloidal gold solution while stirring on a magnetic stirrer, and react for 30 minutes. Add 10% BSA to a final concentration of 1%, and gently stir for 10 minutes. Centrifuge at 45,000 rpm for 30 min at 4° C., discard the supernatant and the resulting precipitate is the purified colloidal gold-labeled monoclonal antibody. Spray this colloidal gold-labeled monoclonal antibody on the gold label pad, spray the KIVPGVPD-BSA conjugate on the NC film as a test line, spray goat anti-mouse IgG on NC membrane as a quality control line, then adhere the sample pad (glass fiber), gold label pad (glass fiber), NC film (including quality control line and test line) and the absorbent pad to a PVC bottom plate in sequence.

2. Colloidal Gold Test Strip Prepared by Peptide GRDGDQGPV

Dissolve 10 mg of BSA in 0.01 mol/L PBS (pH 7.4)(solution 1). Dissolve 4 mg of purified GRDGDQGPV peptide fragment in 0.01 mol/L PBS (pH 7.4)(solution 2). Dissolve 4 mg of 1-(3-dimethylaminopropyl)-3-ethylcarbodiimide hydrochloride (EDC) in 0.5 ml of 0.01 mol/L PBS (pH 7.4)(solution 3). Add solution 2 to solution 1, mix well in ice bath for 30 min, centrifuge to remove precipitation, then drop it into solution 3, continue ice bath for 15 min, and stir at room temperature for 5 min. Dialysis in 0.01 mol/L PBS (pH7.4) for 24 h, during this period, change the dialysate twice, and carry out centrifugation to remove precipitation to obtain the conjugate of the peptide fragment GRDGDQGPV and bovine serum albumin; Immunize BALB/C mice with GRDGDQGPV-BSA; The spleen lymphocytes of immunized mice and myeloma cell (SP2/0) is fused under the promotion of PEG, and the hybridoma cell line stably secreting GRDGDQGPV-BSA monoclonal antibody is screened; The hybridoma cell line is grown in increments, and the obtained culture solution is purified by the caprylic acid-saturated ammonium sulfate method to obtain monoclonal antibodies, which is frozen and stored at −20° C. for later use.

Put an Erlenmeyer flask containing 100 ml of 0.01% chloroauric acid solution on a magnetic stirrer to heat and stir. Quickly add 2 ml of 1% trisodium citrate after boiling, and continue to boil for 5 minutes after the solution turns to wine red. After cooling to room temperature, make up to 100 ml with ultrapure water to obtain a colloidal gold solution with a gold particle diameter of about 10 nm. Adjust the pH value of the obtained 100 ml colloidal gold solution to 8.2 with 20 mol/L borate buffer, slowly add 45 μg of monoclonal antibody GRDGDQGPV-BSA per ml of colloidal gold solution while stirring on a magnetic stirrer, and react for 30 minutes. Add 10% BSA in gold standard solution to a final concentration of 1%, and gently stir for 10 minutes. Centrifuge at 45,000 rpm for 30 min at 4° C., discard the supernatant and the resulting precipitate is the purified colloidal gold-labeled monoclonal antibody. Spray this colloidal gold-labeled monoclonal antibody on the gold label pad, spray the GRDGDQGPV-BSA conjugate on the NC film as a test line, spray goat anti-mouse IgG on NC membrane as a quality control line, then adhere the sample pad (glass fiber), gold label conjugate pad (glass fiber), NC film (including quality control line and test line) and the absorbent pad to a PVC bottom plate in sequence.

### Embodiment 3

Detecting Stichopus Oligopeptide by Colloidal Gold Immunoassay

S1. Dissolve the sample with distilled water to 1 mg/ml, and add 3 drops to the sample pad of the colloidal gold test strip prepared in Embodiment 2;

S2. After the quality control line develops color, observe whether the detection line changes from colorless to red; if the detection line turns red, the sample to be tested is not stichopus oligopeptide; if the detection line does not change color, the sample to be tested is stichopus oligopeptide.

### Embodiment 4

Sensitivity Detection of Colloidal Gold Test Strip

According to the method described in Embodiment 3, stichopus oligopeptide with 8 gradients, respectively 0 μg/ml, 5 μg/ml, 10 μg/ml, 20 μg/ml, 40 μg/ml, 80 μg/ml, 100 μg/ml, and 1000 μg/ml are detected with the detection strip described in Embodiment 2, and the detection line is observed with naked eyes three times. Results are as shown in icon 1, it's found that a deep red band appears on the detection line when the concentration of stichopus oligopeptide is 0 μg/ml. With the increase of the concentration, the color of the detection line gradually weakens. When the concentration of stichopus oligopeptide increases to 20 μg/ml, there is no red band on the detection line. When the concentration of stichopus oligopeptide increases to 1000 μg/ml, there is no red band on the detection line and red band appears on the quality control line. Therefore, it is judged that the detection sensitivity of the test strip is 20 μg/ml.

TABLE 1
Sensitivity detection results of colloidal
gold test strips in the present invention

| Stichopus | | | |
|---|---|---|---|
| oligopeptide/ | Parallel sample | | |
| μg/ml | 1 | 2 | 3 |
| 0 | − | − | − |
| 5 | − | − | − |
| 10 | − | − | − |
| 20 | + | + | + |
| 40 | + | + | + |
| 80 | + | + | + |
| 1000 | + | + | + |

Note:
“+” means positive,
“−” means negative

### Embodiment 5

Specificity Detection of Colloidal Gold Test Strip

Prepare stichopus oligopeptide, soybean oligopeptide, marine fish oligopeptide and oyster oligopeptide respectively into 1 mg/ml samples, and detect them according to the method described in Embodiment 3. The results are as shown in Table 2. The results show that there is no color in the detection line of stichopus oligopeptide samples, and while a red band appears in the quality control line. Red bands appear in the sample detection lines of soybean oligopeptide, marine fish oligopeptide and oyster oligopeptide, and red bands also appear in the quality control line, which indicates that the test strip has good specificity.

TABLE 2
Specificity detection results of colloidal
gold test strips in the present invention

| | Stichopus | Soybean | Marine fish | Oyster |
|---|---|---|---|---|
| Sample | oligopeptide | oligopeptide | oligopeptide | oligopeptide |
| Result | + | − | − | − |

Note:
“+” means positive,
“−” means negative

It should be noted that the colloidal gold labeled monoclonal antibody in Embodiment 2 may also be a mixture of a specific anti-KIVPGVPD monoclonal antibody and a specific anti-GRDGDQGPV monoclonal antibody; preferably, the mass ratio of the specific anti-KIVPGVPD monoclonal antibody to the specific anti-GRDGDQGPV monoclonal antibody is 1:1.

It should be noted that when the claims of the present invention involve numerical ranges, it should be understood that two endpoints of each numerical range and any value between the two endpoints can be selected. For the sake of avoiding elaboration, the present invention describes the preferred embodiments.

Although preferred embodiments of the present invention have been described, additional changes and modifications may be made to these embodiments once those skilled in the art have become aware of the basic inventive concepts. As such, that append claims are intended to be interpreted as including the preferred embodiments and all alterations and modification falling within the scope of the invention.

It will be apparent to those skilled in the art that various modifications and variations can be made in the present invention without departing from the spirit and scope of the invention. Thus, if these modifications and variations of the present invention fall within the scope of the claims of the present invention and their equivalent technologies, the present invention is also intended to include these modifications and variations.

What is claimed is:

1. A peptide fragment used for preparing a colloidal gold test strip for detection of stichopus oligopeptide, the peptide fragment comprising SEQ ID NO: 1 or SEQ ID NO: 3.

2. A colloidal gold test strip, comprising a sample pad, a gold label pad, an NC membrane, an absorbent pad and a PVC bottom plate, wherein the gold label pad is coated with a monoclonal antibody of SEQ ID NO: 1 or SEQ ID NO: 3 labeled with colloidal gold.

3. The colloidal gold test strip according to claim 2, wherein the NC membrane sequentially comprises a detection line and a quality control line according to the direction of sample chromatography, the aforesaid detection line is coated with a conjugate of peptide fragment SEQ ID NO: 1 or SEQ ID NO: 3 and bovine serum albumin; and the quality control line is coated with sheep anti-mouse secondary antibody.

4. The colloidal gold test strip according to claim 2, wherein the monoclonal antibody is prepared according to the following method:
take chloroauric acid solution and heat it, add trisodium citrate after boiling, and continue to boil for more than 5 minutes after the solution turns to wine red; After cooling to room temperature, add ultrapure water to obtain a colloidal gold solution; Adjust the pH value of the obtained colloidal gold solution to 7.8-8.5 with borate buffer, stir the colloidal gold solution and add monoclonal antibody, react for 20-40 minutes, add BSA, and centrifuge after stirring.

5. A method for detection of stichopus oligopeptide by using the colloidal gold test strip according to claim 2, wherein it comprises the following steps: 1) dissolve a sample in distilled water and drop it on the sample pad; 2) observe whether that detection line changes from colorless to red after the quality control line develops color; If the detection line turns red, the sample to be detected is not stichopus oligopeptide; If the detection line does not change color, the sample to be detected is stichopus oligopeptide.


SEQUENCE LISTING (4 sequences)

SEQ ID NO: 1 - protein, 8 aa, Apostichopus japonicus
KIVPGVPD

SEQ ID NO: 2 - protein, 881 aa, Apostichopus japonicus
MGVFATGLDWELEAPNPIPGVPTDITIDASALFTPTSRNLEGDDLWRLSIFGSDNGRGSGPRVNENRQILDQDQASTTFTPGEPVDIDGITTEFDVGSVGCTDELTHVCVEFTRGDDPNPEYSFRTVDPLLGTEESIISCKELPCGAKAIFTELNADLTEPDVIIEDQPNTLVVDLTAVRHPTSTAVAGEDLWKVKSYFSPRDDGRGPKYDPKEEILTPAYKDTPLLYDQDLDFEGISIEADLSGVTCSQASYFCMEMEKDAAASIDYEFGTIPEQDPFVTCVDITDRCKVSIYHYGIVLFNPPRCGRHNLDFTREVPDFIPSGQPVPVTIDATIDMEPYSRGVSGACLWKVNMFGSRNNDGSGARQPLSSQILDSYEAAIPLSPGGPLYFENLQADFDVSQLGCGEIGYLCLEFTQCDTPNPDFSFEASDGGESIVKCQAENCRAVFIDDFTTNPIDIDLVEGRRSNPVNFQGTASSTDESGTVPSGFDLWTVSFWGSKKPNGKGEQQNPQEQVLDPYYASTPLYNPGDNIDMSRIFSNFDMDGLQCDDVPYLCAKFNRDPNSRPPFEMEAVPDERVLTSCFPVPEDSCKGVIATDLDFDIEFDKIVPGVPDDVTFDIDLSTLYTSAGADGNNLWRVGFFTSPDEIGSVKSNYDPQILTRSSSSTDLEPGEALKLDNVNTQVDLTGLGCEEDMKFVCLEFAKGQRANPDFKFEVSGGGDVLVKCIEQECEKPIVVTELEVQPYGTITVTEGNPTNRVLFSLTGVTDKDESGKAIGDDLWDLDTWGSTTEDCSGTKHNPKTQNNLQQSQMDRDVYEGQDVSLGAVDTEFDMSGLNCEEVKYFCAHLKKNAKAEPDFEFETKPDESAAIASFAVDCKGKGNL

SEQ ID NO: 3 - protein, 9 aa, Apostichopus japonicus
GRDGDQGPV

SEQ ID NO: 4 - protein, 1813 aa, Apostichopus japonicus
MISGVVLTDMNWDFTVQGPKKPNGSELLGINIQVDPARTSASVDGKRLWRAALFRSSSPNGDGPRVNSKMQILNKYQASRPLEGGGQTMRIDGVQAEFDMDEFAGCSNDYQYLCLEFTKNRKPKVNFRMTIEGGDRSIIKCKFQECMRGIELTDFEAEPGRPVELMEGKSENSVTFRLTATPSLQSGGIQGFDLWNLNVFGSLNPFGAGERINQVSEVLAPNQRSTGLFPPSIMNFGHVQHYFNMTGVECGDIMYLCAELVQGAEPNPEFQLNAIPDERVLIDCFNVHCLGIVIRETKLDLYNGGGLLDRDVDTNHLSFGVQILAGMESGDAVGDMLWRMEAYISNDYAGYGTRHVLKRQLLNIEQSGYDIHSGERLEFNNLATMLERDQLSCEEQLYLCVELMRGNDKIKFMGSRPGSLRDCEELNCAERPKPVVERFSFFLLHVVKFNLLHGLTGPKGPKGEPGQTPVIGSGDLIPGPPGPPGGRGGPGDRGPLGPKGNIGIPGPRGGSGADGVPGLPGPPGPPGPPGPSGQLTAEAQVSQQYKGPVMPAGLGYPQAMMAGEPGQRGPPGQAGLRGPAGLTGAPGEPGEQGPTGPVGLTGPAGSPGRPGSAGKDGANGNPGPAGPSGPPGPAGQNGFPGPSGPKGHRGYSGRAGQKGERGDDGERGNDGAAGSIGAPGPAGPMGSPGERGRTGPSGDTGPAGSDGAPGAQGPPGSAGIPGAPGVPGSSGPKGDAGSPGATGPQGQQGSRGERGPEGQQGQAGLQGAPGMNGNPGAKGASGAPGAQGPIGFSGLRGPNGAPGSQGPAGSKGDQGVAGAPGEQGEEGPTGLPGDVGPQGPPGANGASGERGPRGSTGPAGPQGPAGDRGQLGAPGSIGPAGAAGAKGETGARGGPGSPGSKGSRGEPGRNGDPGLPGTRGLSGNPGKQGKDGSPGPPGPPGENGRQGATGATGQRGMPGAQGLTGAPGAQGDRGGDGQVGPPGPAGPQGEVGDRGNPGPAGAPGSPGVAGERGAPGLQGPQGQQGPAGLNGPQGESGAPGDAGPQGETGAPGNPGDRGERGGPGDRGPPGPSGRPGGRGVSGRAGQDGATGAPGPKGEQGPPGGPGLGGQQGPRGPPGPSGSRGPPGEDGDEGRPGPTGPAGPRGSIGPIGPTGITGSPGEKGDQGSPGPAGAQGARGDAGARGANGPAGPQGFPGPAGRPGSPGARGETGPSGIRGETGPAGAAGATGDPGAQGPAGAVGAQGERGEAGNTGPQGPVGNPGIGGPQGNQGPPGPAGQTGPSGAPGAPGERGDPGVAGRDGDQGPVGAPGASGEKGDRGSDGDIGSAGPAGPPGPPGASGPLGIAGSMGPRGPPGAPGGPGEQGATGSPGSQGNRGSPGAIGAPGLLVQLVPMDKMVKMVVMVPKAQPDNVVRRETLVQPVTQDLKVAQDLRGLLDKMVPQESVVKLVPQAPLDPKDLLDNVDLLDQLAQLDLLVQLVNVVHLAHRVLKVIKDPLVSKGSPGDTGVPGPPGTAGERGSPGNRGAAGMDGATGPRGFDGPEGPRGPPGSEGRQGSQGPPGPPGPPGPPGTVQGFTYSQYPNQVVSGQDKGPSPYYYGDDANVDAKNKEPEILATLKDINNRFKALKQPSGDSKLHATRSCRNLFLDHPDKESGIYWVDPNLGCTDDAFQAFCDKTNKATCLTPETHLVTNATWYTGKPKRIYYSSMRGGEKFTYAEKSQLVFLRLLSISAKQSVTFYCRNVAANLEFLNSREDVMRDYQILENTCQSQSDSWGRVVLDYETDITDNLPFEDFSVGDLGADNQEFGLDMGKVCFA